(19) United States
(12) Patent Application Publication
(10) Pub. No.: US 2014/0107776 A1
(43) Pub. Date: Apr. 17, 2014

(54) PROSTHESES FOR OCULAR DRUG DELIVERY AND OTHER TREATMENTS FOR GLAUCOMA, MACULAR DEGENERATION, AND OTHER EYE DISORDERS OR DISEASES

(71) Applicant: REFOCUS GROUP, INC., Dallas, TX (US)

(72) Inventors: Douglas C. Williamson, Coppell, TX (US); Gary A. Richardson, Rochester, NY (US); John R. Samples, Highlands Ranch, CO (US)

(73) Assignee: REFOCUS GROUP, INC., Dallas, TX (US)

(21) Appl. No.: 14/133,453

(22) Filed: Dec. 18, 2013

Related U.S. Application Data
(63) Continuation-in-part of application No. 13/654,249, filed on Oct. 17, 2012.
Application No. 13/654,249 is a continuation-in-part of application No. 11/827,382, filed on Jul. 11, 2007, now Pat. No. 8,409,277.
(60) Provisional application No. 60/819,995, filed on Jul. 11, 2006.

(51) Int. Cl. A61F 2/14

(52) U.S. Cl. CPC A61F 2/14
USPC 623/4.1

(57) ABSTRACT

A scleral prosthesis includes an elongated body that is narrower in a middle and wider at opposing first and second ends. The body includes multiple first portions that form the first end of the body, where the first portions separated lengthwise along the body. The first portions are biased to maintain separation without external interference but are configured to be pushed towards each other in order to narrow a width of the first end. The body could also include second portions that form the second end of the body. The body could further include a cavity configured to receive a material and an eluting surface configured to release the material from the cavity. In addition, the body could include a cavity configured to receive a material and a protrusion that extends away from the scleral prosthesis, where the protrusion is configured to release the material from the cavity.

### CROSS-REFERENCE TO RELATED APPLICATIONS AND PRIORITY CLAIM

This application claims priority under 35 U.S.C. §120 as a continuation-in-part of U.S. patent application Ser. No. 13/654,249 filed on Oct. 17, 2012, which is a continuation-in-part of U.S. patent application Ser. No. 11/827,382 filed on Jul. 11, 2007 (now U.S. Pat. No. 8,409,277), which claims priority to U.S. Provisional Patent Application No. 60/819,995 filed on Jul. 11, 2006. All of these patent applications are hereby incorporated by reference in their entirety.

### TECHNICAL FIELD

This disclosure is generally directed to eye implants. More specifically, this disclosure relates to prostheses for ocular drug delivery and other treatments for glaucoma, macular degeneration, and other eye disorders or diseases.

### BACKGROUND

It is possible to treat presbyopia, glaucoma, and other eye disorders or diseases by implanting scleral prostheses within the sclera of a patient's eye. For each individual scleral prosthesis, an incision can be made in the sclera of the patient's eye, such as near the plane of the equator of the crystalline lens. The incision can then be extended under the surface of the sclera to form a scleral “tunnel,” and a scleral prosthesis can be placed within the tunnel. Alternatively, an incision can be made in the sclera of the patient's eye to form a scleral “pocket,” a scleral prosthesis can be inserted into the pocket, and the incision can be closed (such as with a suture). One or multiple scleral prostheses may be implanted in a patient's eye to (among other things) treat presbyopia, glaucoma, ocular hypertension, elevated intraocular pressure, macular degeneration, or other eye disorders or diseases.

Techniques, scleral prostheses, and tools for treating presbyopia, glaucoma, and other eye disorders or diseases are described more fully in the following patents and patent applications, which are hereby incorporated by reference:
(1) U.S. Pat. No. 6,007,578 entitled “Scleral Prosthesis for Treatment of Presbyopia and Other Eye Disorders” issued on Dec. 28, 1999;
(2) U.S. Pat. No. 6,280,468 entitled “Scleral Prosthesis for Treatment of Presbyopia and Other Eye Disorders” issued on Aug. 28, 2001;
(3) U.S. Pat. No. 6,299,640 entitled “Scleral Prosthesis for Treatment of Presbyopia and Other Eye Disorders” issued on Oct. 9, 2001;
(4) U.S. Pat. No. 6,991,650 entitled “Scleral Expansion Device Having Duck Bill” issued on Jan. 31, 2006;
(5) U.S. Pat. No. 7,416,560 entitled “Scleral Prosthesis for Treatment of Presbyopia and Other Eye Disorders” issued on Aug. 26, 2008;
(6) U.S. Pat. No. 7,780,727 entitled “Scleral Prosthesis for Treatment of Presbyopia and Other Eye Disorders” issued on Aug. 24, 2010;
(7) U.S. Pat. No. 7,785,367 entitled “Scleral Prosthesis for Treatment of Presbyopia and Other Eye Disorders” issued on Aug. 31, 2010;
(8) U.S. Pat. No. 7,927,372 entitled “Scleral Prosthesis Having Crossbars for Treating Presbyopia and Other Eye Disorders” issued on Apr. 19, 2011;
(9) U.S. Pat. No. 8,337,550 entitled “Scleral Prosthesis for Treating Presbyopia and Other Eye Disorders and Related Devices and Methods” issued on Dec. 25, 2012;
(10) U.S. Pat. No. 5,354,331 entitled “Treatment of Presbyopia and Other Eye Disorders” issued on Oct. 11, 1994;
(11) U.S. Pat. No. 5,465,737 entitled “Treatment of Presbyopia and Other Eye Disorders” issued on Nov. 14, 1995;
(12) U.S. Pat. No. 5,489,299 entitled “Treatment of Presbyopia and Other Eye Disorders” issued on Feb. 6, 1996;
(13) U.S. Pat. No. 5,503,165 entitled “Treatment of Presbyopia and Other Eye Disorders” issued on Apr. 2, 1996;
(14) U.S. Pat. No. 5,529,076 entitled “Treatment of Presbyopia and Other Eye Disorders” issued on Jun. 25, 1996;
(15) U.S. Pat. No. 5,722,952 entitled “Treatment of Presbyopia and Other Eye Disorders” issued on Mar. 3, 1998;
(16) U.S. Pat. No. 6,197,056 entitled “Segmented Scleral

Band for Treatment of Presbyopia and Other Eye Disorders” issued on Mar. 6, 2001;
(17) U.S. Pat. No. 6,579,316 entitled “Segmented Scleral Band for Treatment of Presbyopia and Other Eye Disorders” issued on Jun. 17, 2003;
(18) U.S. Pat. No. 6,926,727 entitled “Surgical Blade for Use with a Surgical Tool for Making Incisions for Scleral Eye Implants” issued on Aug. 9, 2005;
(19) U.S. Pat. No. 7,189,248 entitled “System and Method for Making Incisions for Scleral Eye Implants” issued on Mar. 13, 2007;
(20) U.S. Pat. No. 7,824,423 entitled “System and Method for Making Incisions for Scleral Eye Implants” issued on Nov. 2, 2010;
(21) U.S. Pat. No. 7,854,742 entitled “System and Method for Making Incisions for Scleral Eye Implants” issued on Dec. 21, 2010;
(22) U.S. Pat. No. 8,361,098 entitled “Surgical Blade for Use with a Surgical Tool for Making Incisions for Scleral Eye Implants” issued on Jan. 29, 2013;
(23) U.S. Pat. No. 8,083,759 entitled “Apparatuses and Methods for Forming Incisions in Ocular Tissue” issued on Dec. 27, 2011;
(24) U.S. Pat. No. 7,909,780 entitled “System and Method for Determining a Position for a Scleral Pocket for a Scleral Prosthesis” issued on Mar. 22, 2011;
(25) U.S. Pat. No. 8,201,942 entitled “System and Method for Identifying a Position to Insert a Scleral Prosthesis into an Eye” issued on Jun. 19, 2012;
(26) U.S. Patent Publication No. 2013/0041394 entitled “Apparatus and Method for Forming Incisions in Ocular Tissue” filed on Aug. 8, 2011;
(27) U.S. Patent Publication No. 2012/0226107 entitled “Apparatus and Method for Securing Ocular Tissue” filed on Mar. 6, 2012;
(28) U.S. Patent Publication No. 2012/0078281 entitled “Apparatuses and Methods for Forming Incisions in Ocular Tissue” filed on Dec. 7, 2012;
(29) U.S. Patent Publication No. 2012/0078280 entitled “Apparatuses and Methods for Forming Incisions in Ocular Tissue” filed on Dec. 7, 2012;
(30) U.S. Patent Publication No. 2012/0035637 entitled “Apparatuses and Methods for Forming Incisions in Ocular Tissue” filed on Oct. 14, 2011;
(31) U.S. Patent Publication No. 2010/0152848 entitled “Artificial Intraocular Lens, Altered Natural Crystalline Lens, or Refilled Natural Crystalline Lens Capsule with One or More Scleral Prostheses for Improved Performance” filed on Nov. 19, 2009; and
(32) U.S. Patent Publication No. 2008/0091266 entitled “Scleral Prosthesis for Treating Presbyopia and Other Eye Disorders and Related Devices and Methods” filed on Jul. 11, 2007.

### SUMMARY

This disclosure provides prostheses for ocular drug delivery and other treatments for glaucoma, macular degeneration, and other eye disorders or diseases.

In a first embodiment, a prosthesis includes an elongated body that is narrower in a middle and wider at opposing first and second ends. The body includes multiple first portions that form the first end of the body, where the first portions are separated lengthwise along the body. The first portions are biased to maintain separation without external interference but are configured to be pushed towards each other in order to narrow a width of the first end.

In a second embodiment, a prosthesis includes an elongated body that is narrower in a middle and wider at opposing first and second ends. The body includes multiple first portions that form the first end of the body, where the first portions are separated lengthwise along the body. The first portions are biased to maintain separation without external interference but are configured to be pushed towards each other in order to narrow a width of the first end. The prosthesis also includes an insert configured to be placed between the first portions to maintain the separation of the first portions.

In a third embodiment, a system includes a prosthesis configured to be implanted into a patient's eye. The system also includes a reservoir configured to be coupled to the prosthesis. The reservoir includes a cavity configured to receive a material. At least one of the prosthesis and the reservoir is configured to release the material into a patient's eye.

Other technical features may be readily apparent to one skilled in the art from the following figures, descriptions, and claims.

### BRIEF DESCRIPTION OF THE DRAWINGS

For a more complete understanding of this disclosure, reference is now made to the following description, taken in conjunction with the accompanying drawing, in which:

FIGS. 1A and 1B illustrate example scleral prostheses in accordance with this disclosure;

FIGS. 2A through 2E illustrate example reservoirs (used with or without a scleral prosthesis) in accordance with this disclosure;

FIGS. 3A through 3E illustrate example uses of a scleral prosthesis (with and without a reservoir) in accordance with this disclosure;

FIGS. 4A and 4B illustrate another example scleral prosthesis in accordance with this disclosure; and

FIGS. 5A and 5B illustrate example methods for inserting and using a scleral prosthesis in a patient's eye in accordance with this disclosure.

### DETAILED DESCRIPTION

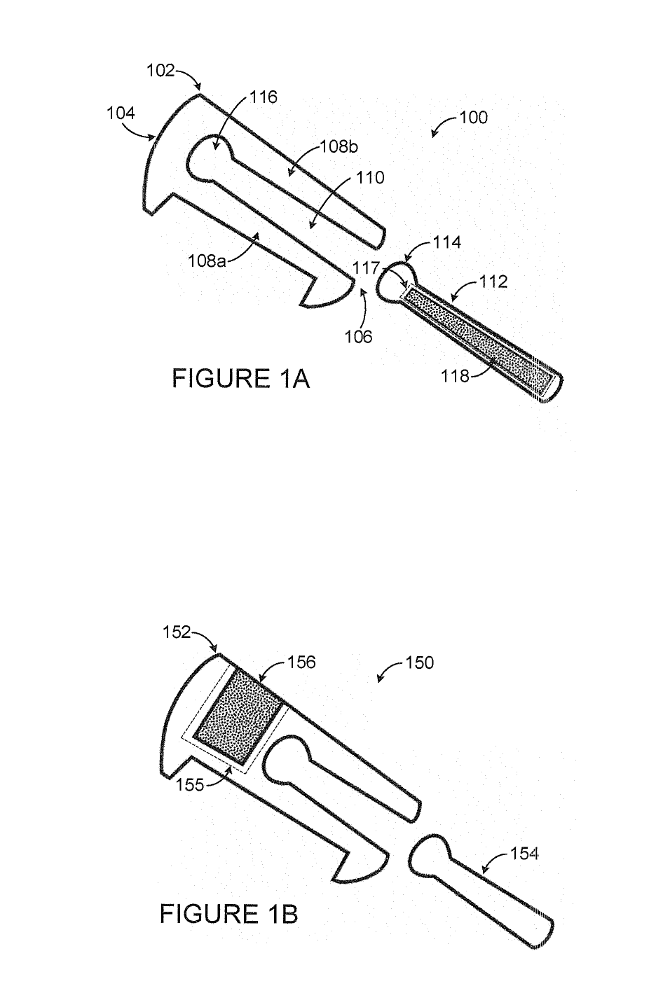

FIGS. 1 through 5B, discussed below, and the various embodiments used to describe the principles of the present invention in this patent document are by way of illustration only and should not be construed in any way to limit the scope of the invention. Those skilled in the art will understand that the principles of the present invention may be implemented in any type of suitably arranged device or system.

FIGS. 1A and 1B illustrate example scleral prostheses in accordance with this disclosure. The embodiments of the scleral prostheses shown in FIGS. 1A and 1B are for illustration only. Other embodiments of the scleral prostheses could be used without departing from the scope of this disclosure.

As shown in FIG. 1A, a scleral prosthesis 100 has a main body 102 with two opposing ends 104-106. Each end 104-106 is wider than a middle portion of the body 102. One end 106 of the body 102 is split or divided into multiple legs or portions 108a-108b. In this example, the portions 108a-108b are separated lengthwise along at least half of the prosthesis 100 (approximately 75-80% of the length in FIG. 1A, although other lengths such as about 50% or more could be used). The portions 108a-108b can be pushed together, such as at the end 106, to narrow the cross-sectional area of the end 106 of the body 102. This may allow the scleral prosthesis 100 to be inserted more easily into a scleral tunnel or to be inserted into a smaller scleral tunnel in a patient's eye.

The scleral prosthesis 100 also includes an opening 110, which allows an insert 112 to be placed between the portions 108a-108b of the end 106. The insert 112 could be permanently or removably placed between the portions 108a-108b of the end 106. For example, the insert 112 could be placed between the portions 108a-108b of the end 106 after the prosthesis body 102 has been implanted in a scleral tunnel in a patient's eye. The insert 112 could later be removed, such as to facilitate removal of the prosthesis 100 from the scleral tunnel.

The insert 112 may generally help to stabilize the prosthesis 100. For example, the portions 108a-108b of the body 102 could be biased to maintain their separation from one another absent external interference. The insert 112 could help to prevent the portions 108a-108b of the body 102 from being pushed towards each other (which, if it occurred, might allow the body 102 to slip through the scleral tunnel). The insert 112 could also function to reduce or prevent rotation of the prosthesis 100 within the scleral tunnel. For instance, the insert 112 may help to ensure that the end 106 of the body 102 maintains a desired width and therefore remains wide enough to prevent the prosthesis 100 from rolling over once implanted in the scleral tunnel. Moreover, the insert 112 can be inserted into the body 102 after the body 102 has been implanted. This enables the portions 108a-108b of the body 102 to be pushed together during implantation.

The insert 112 could be attached or coupled to the body 102 in any suitable manner. For example, the insert 112 could have one or more structures that engage one or more corresponding structures of the body 102. In this example, the insert 112 includes a head 114 that is wider than the adjacent portion of the insert 112. The head 114 snaps or otherwise fits within a portion 116 of the opening 110 in the body 102. The insert 112 could also be attached to the body 102 using sutures, adhesive, welding, or any other suitable technique.

In this example, the prosthesis 100 does not have a symmetrical cross-section. Instead, the prosthesis 100 has one side that is relatively flat along the entire length of the prosthesis 100. This is for illustration only, and the prosthesis 100 could have any suitable symmetrical or asymmetrical cross-section. Other example prostheses that could be used are provided in U.S. Patent Publication No. 2008/0091266, which was incorporated by reference above. For instance, another example prosthesis could include legs on both ends of the prosthesis, where one set of legs is separated along at least about half of the total length of the prosthesis and another set of legs is separated along no more than about a quarter of the total length of the prosthesis. Also, the prosthesis 100 may have any suitable shape, such as a convex upper surface and a flat or concave lower surface. Again, this is for illustration only. The prosthesis 100 could generally have any suitable shape for facilitating the treatment of presbyopia, glaucoma, or other eye disorders or diseases.

In FIG. 1A, the insert 112 includes an internal cavity 117 storing at least one medication or other material(s) and a membrane or other eluting surface 118. The cavity 117 represents any suitable structure capable of storing at least one medication or other material(s) for later dispensing. The eluting surface 118 acts as a restrictor over the cavity 117 so that the material(s) can move from the prosthesis 100 into the patient's eye, thereby dispensing the material(s) (possibly in a slow or time-release fashion). The internal cavity 117 of the insert 112 could have any suitable size and shape, such as a size and shape that do not compromise the structural integrity of the insert 112. Also, the eluting surface 118 could be formed from any suitable material(s), such as a fibrous or porous material or membrane. The eluting surface 118 could further be implemented by holes, slits, or other openings in the body of the insert itself.

As shown in FIG. 1B, a scleral prosthesis 150 has a similar structure as the prosthesis 100, with a main body 152 and an insert 154 placed into the main body 154. In this embodiment, the main body 152 includes an internal cavity 155 storing at least one medication or other material(s) and a membrane or other eluting surface 156. Again, the eluting surface 156 represents any suitable surface, material, or other membrane through which at least one medication or other material(s) can move from the prosthesis 150 into the patient's eye. The internal cavity 155 of the main body 152 could have any suitable size and shape, such as a size and shape that do not compromise the structural integrity of the main body 152. Also, the eluting surface 156 could be formed from any suitable material(s), such as a fibrous or porous material or membrane. The eluting surface 156 could further be implemented by holes, slits, or other openings in the body of the prosthesis itself.

In particular embodiments, the scleral prostheses 100 and 150 in FIGS. 1A and 1B may be formed from polymethyl methacrylate (“PMMA”), polyether-ether ketone (“PEEK”), or other suitable material(s). Also, the prostheses 100 and 150 could have any suitable size, shape, and dimensions, and prostheses of different sizes, shapes, or dimensions could be provided. For example, different-sized prostheses could have different lengths, such as lengths of 3.5, 4.0, 4.5, 5.0, and 5.5 millimeters or longer.

In these example embodiments, the prostheses 100 and 150 facilitate more efficient or effective delivery of one or more medications or other material(s) to a patient's eye. For example, the prostheses 100 and 150 can be used to distribute a medication or other material(s) directly into the patient's scleral tissue. Moreover, as described in more detail below, the internal cavity 117, 155 of the insert or prosthesis body could be refillable, enabling the prostheses 100 and 150 to distribute a medication or other material(s) over an extended period of time. This may allow, for example, a doctor or other personnel to repeatedly refill the prostheses 100 and 150 with medication or other material(s), reducing or eliminating the need to inject the medication or other material(s) directly into the patient's eye.

In addition, the prostheses 100 and 150 provide two different mechanisms for treating presbyopia, glaucoma, and other eye disorders or diseases. First, the prostheses 100 and 150 could be inserted into a patient's eye to treat an eye disorder or disease (as described in the incorporated patent documents). Second, the medication(s) or other material(s) distributed by the prostheses 100 and 150 could be used to treat the same eye disorder or disease or a different eye disorder or disease. As a result, the prostheses 100 and 150 may help to provide more efficient and effective treatment for one or more eye disorders or diseases in a patient, and it can be done in a manner that is more acceptable to the patient.

Although FIGS. 1A and 1B illustrate examples of scleral prostheses 100 and 150, various changes may be made to FIGS. 1A and 1B. For example, a scleral prosthesis could have any suitable size, shape, and dimensions. Also, a scleral prosthesis could have any suitable number of cavities and eluting surfaces, whether on the prosthesis' main body, insert, or other element. Further, a scleral prosthesis could be formed from a single integrated component and need not include any type of insert. In addition, FIGS. 1A and 1B illustrate example scleral prostheses that support ocular delivery of drugs or other materials. This functionality could be used with any other scleral prosthesis (including any of the various scleral prostheses disclosed in the patent documents incorporated by reference above).

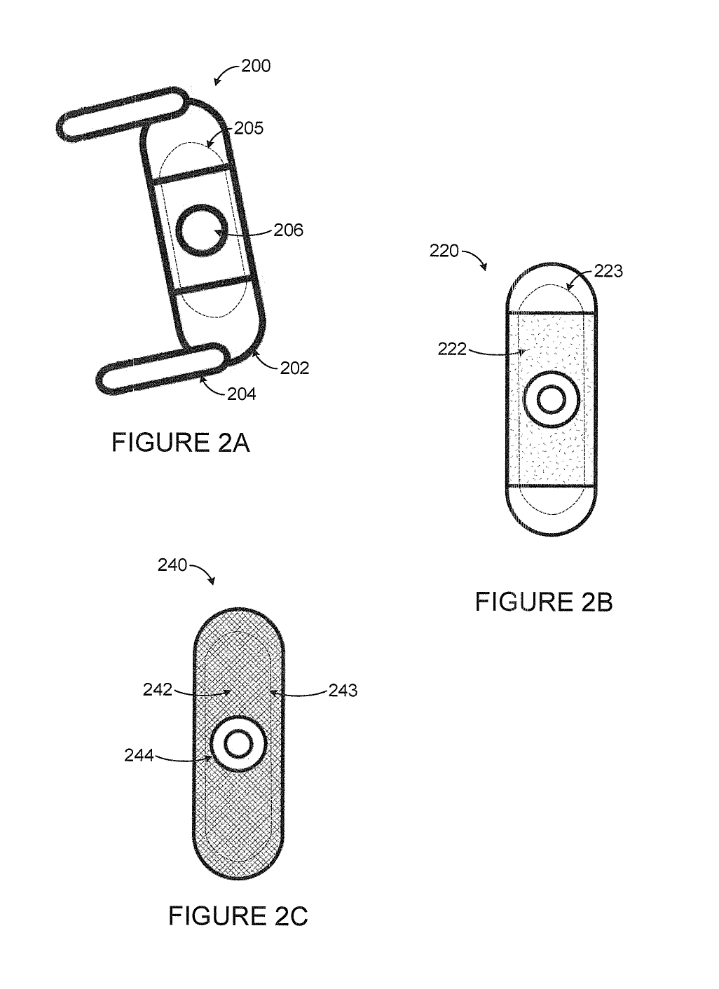

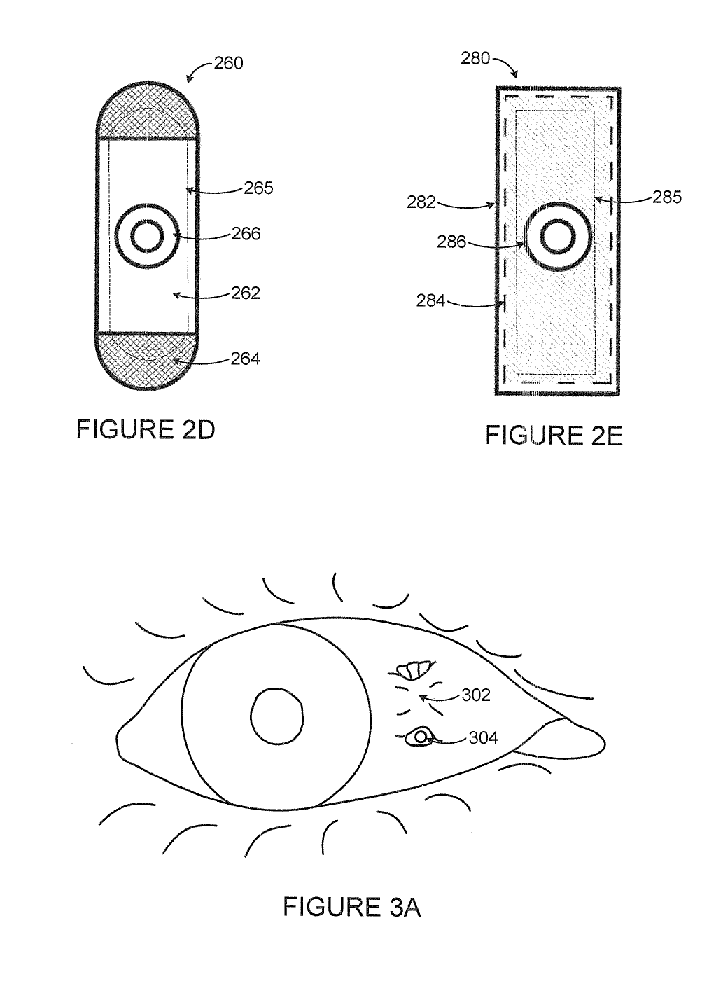

FIGS. 2A through 2E illustrate example reservoirs (used with or without a scleral prosthesis) in accordance with this disclosure. The embodiments of the reservoirs shown in FIGS. 2A through 2E are for illustration only. Other embodiments of the reservoir could be used without departing from the scope of this disclosure.

In FIG. 2A, a reservoir 200 generally represents a structure in which one or more medications or other material(s) can be stored. In this example, the reservoir 200 includes a main storage body 202 and one or more connecting arms 204. The main storage body 202 includes an internal cavity 205 for storing one or more medications. The main storage body 202 could, for example, represent a hard shell surrounding an inner cavity. The connecting arms 204 can be used to secure the reservoir 200 in place in a patient's eye, such as by attaching or securing the reservoir 200 to one or more scleral prostheses implanted in the patient's eye. The body 202 of the reservoir 200 could have any suitable size and shape, and the body 202 could be formed from any suitable material or materials (such as PMMA or PEEK). Also, each connecting arm 204 could have any suitable size and shape and be formed from any suitable material or materials (such as PMMA or PEEK).

The body 202 of the reservoir 200 includes a refill hole 206. The refill hole 206 provides access to the internal cavity 205 of the body 202. This may allow, for example, additional medication or other material(s) to be injected into the body 202 of the reservoir 200, such as by using a hypodermic needle. The refill hole 206 could have any suitable size and shape, and the hole 206 could be formed in the body 202 in any suitable manner. Also, any suitable technique could be used to refill the reservoir 200, and the refill technique is not limited to use with hypodermic needles.

Depending on the implementation, at least one medication or other material(s) stored within the reservoir 200 can be provided to a scleral prosthesis for delivery to a patient's eye. The medication or other material(s) could, for instance, be provided to the scleral prosthesis through one or more of the connecting arms 204.

A similar reservoir 220 is shown in FIG. 2B. In this example, the reservoir 220 has the same general shape as the reservoir 200. In addition, the reservoir 220 includes one or more membranes or other eluting surfaces 222. The eluting surface 222 represents a surface through which at least one medication or other material(s) can move from an internal cavity 223 of the reservoir 220 into the patient's eye, thereby dispensing the medication or other material(s). The eluting surface 222 could be formed from any suitable material(s), such as a fibrous or porous material or membrane. The eluting surface 222 could also be implemented by holes, slits, or other openings in the body of the reservoir itself. Connecting arms 204 may or may not be used with the reservoir 220.

In FIG. 2C, a reservoir 240 includes a flexible body 242, which could be formed from a fibrous or porous material or membrane or other eluting surface. In this example, the body 242 may lack a hard shell or other structure. As a particular example, the flexible body 242 could be formed from one or more materials from ALZA CORPORATION. The reservoir 240 also includes an internal cavity 243 for holding one or more medications or other material(s) and a refill hole 244 for refilling the reservoir 240. Connecting arms 204 may or may not be used with the reservoir 240.

A reservoir 260 in FIG. 2D includes a body 262, which in this example has the form of a cylinder with open ends. The body 262 could, for example, be formed from a hard material such as PMMA or PEEK. The reservoir 260 also includes a fibrous or porous material, membrane, or other eluting surface 264, which is disposed within the body 262 and can elute or otherwise deliver medication or other material(s) into a patient's eye. A refill, hole 266 allows an internal cavity 265 in the reservoir 260 to be refilled. Connecting arms 204 may or may not be used with the reservoir 260.

A reservoir 280 in FIG. 2E includes a body 282, which in this example has the form of a cylinder with closed ends. The body 282 could, for example, be formed from a hard material such as PMMA or PEEK. The reservoir 280 also includes a fibrous or porous material, membrane, or other eluting surface 284, which is disposed within the body 282 and can elute or otherwise deliver medication or other material(s) into a patient's eye. The eluting surface 284 could also be implemented using holes, slits, or other openings in the body 282 (in the sides and/or ends). A refill hole 286 allows an internal cavity 285 of the reservoir 280 to be refilled. Connecting arms 204 may or may not be used with the reservoir 280.

As described in more detail below, various ones of these reservoirs could be used in a stand-alone manner, or a scleral prosthesis could be used to attach or secure a reservoir within a patient's eye. In some embodiments, the reservoir could then use one or more membranes or other eluting surfaces to deliver one or more medications or other material(s) to the patient's eye. This may allow, for example, the reservoir to provide medication or other material(s) to the patient's eye without requiring delivery by the scleral prosthesis. In other embodiments, the reservoir could provide one or more medications or other material(s) to the scleral prosthesis for delivery to the patient's eye, such as by providing the medication or other material(s) to the internal cavity of the insert 112 or body 152 through one or more of the connecting arms 204. In yet other embodiments, both the reservoir and the scleral prosthesis could be used to deliver at least one medication or other material(s) from the reservoir to the patient's eye.

Although FIGS. 2A through 2E illustrate examples of reservoirs (used with or without a scleral prosthesis), various changes may be made to FIGS. 2A through 2E. For example, a reservoir could have any suitable size and shape. Also, a reservoir could have any suitable number of internal cavities and membranes or other eluting surfaces (including none). In addition, FIGS. 2A through 2E illustrate example reservoirs that support ocular delivery of drugs or other materials. This functionality could be used with any other scleral prosthesis (including any of the various scleral prostheses disclosed in the patent documents incorporated by reference above) and is not limited to use with the scleral prostheses 100 and 150 of FIGS. 1A and 1B.

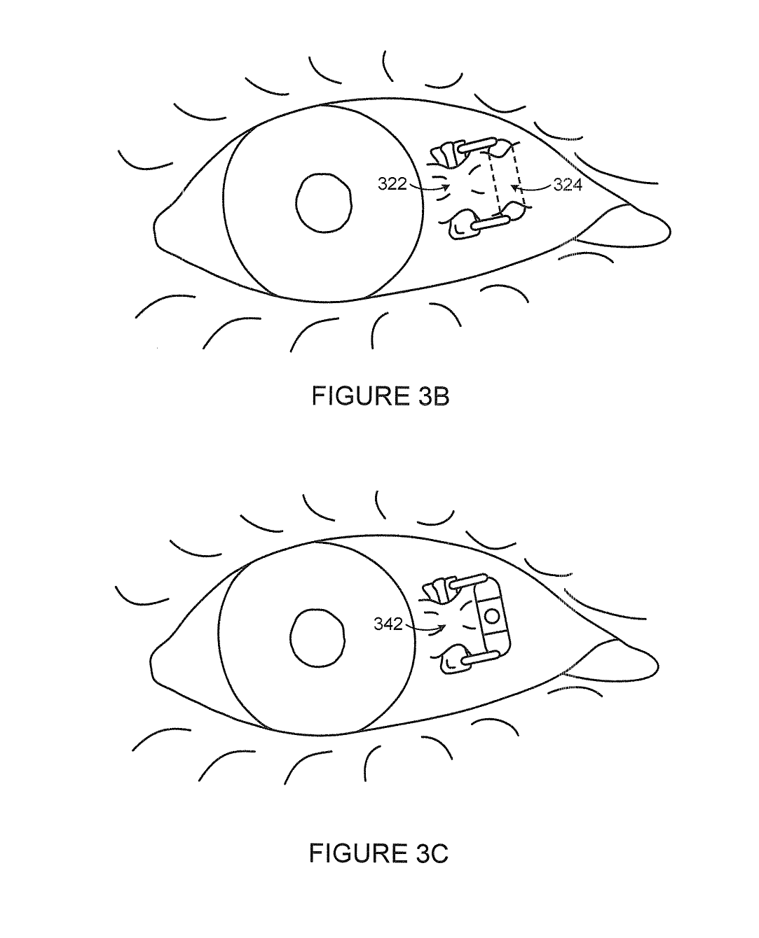

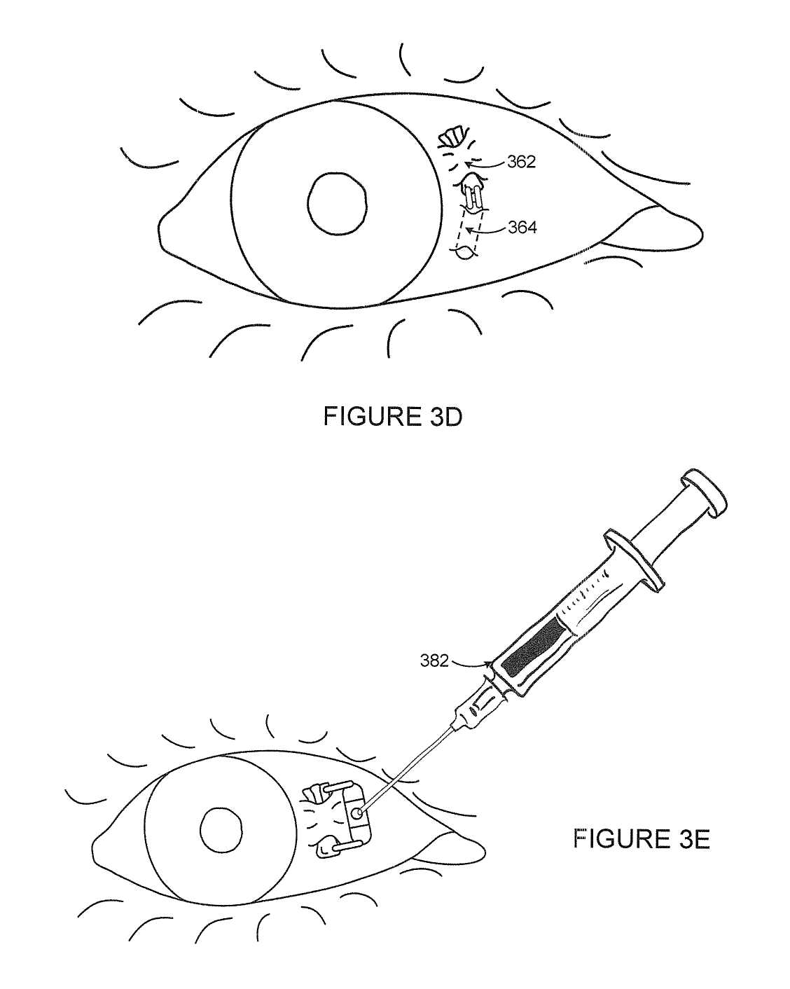

FIGS. 3A through 3E illustrate example uses of a scleral prosthesis (with and without a reservoir) in accordance with this disclosure. The example uses shown in FIGS. 3A through 3E are for illustration only. The scleral prosthesis (and an optional reservoir) could be used in any other suitable manner.

In FIG. 3A, a scleral prosthesis (such as the prosthesis 100 or 150) can be inserted into a scleral tunnel 302 in a patients eye. The scleral tunnel 302 could be formed in any suitable manner, such as by using a surgical tool with a curved or straight cutting blade or with a specialized laser. Additional details regarding specific surgical tools for making scleral tunnels can be found in various ones of the patent documents incorporated by reference above. Once inserted into the scleral tunnel 302, the scleral prosthesis can release medication or other material(s) into the patient's eye, such as glaucoma or macular degeneration medication. Moreover, the mere presence of the scleral prosthesis can help to treat presbyopia, glaucoma, or other eye disorders or diseases. For example, the presence of the scleral prosthesis in the patient's eye could help to increase drainage of aqueous through the trabecular meshwork of the eye, thereby helping to treat glaucoma.

In this particular example, the scleral prosthesis includes a refill hole 304 on at least one end of the prosthesis, which can be used to refill medication or other material(s) in the prosthesis. This may allow, for instance, the scleral prosthesis to be used for material delivery over an extended period of time. This may also help to reduce or avoid the need to inject material into the patient's eye using a hypodermic needle. Note, however, that the refill hole 304 could be placed on the insert that is inserted into a scleral prosthesis body.

In FIG. 3B, a scleral prosthesis (such as the prosthesis 100 or 150) can be inserted into a scleral tunnel 322 in a patients eye. Also, a reservoir (such as any of the reservoirs 200-280) can be inserted into a separate scleral tunnel 324 in the patient's eye and is attached or secured to the scleral prosthesis by the connecting arms. The scleral tunnels 322-324 could be formed in any suitable manner, such as by using a surgical tool with a curved or straight cutting blade or with a specialized laser. Also, the scleral tunnels 322-324 could be separated by any suitable distance, or the scleral tunnels 322-324 could be combined into one larger scleral tunnel in which both the scleral prosthesis and the reservoir are inserted.

In this example, the scleral prosthesis and/or the reservoir could be used to release medication or other material(s) into the patient's eye. Although not shown, since the reservoir is located within a scleral tunnel, the refill hole of the reservoir could be moved to at least one end of the reservoir, thereby allowing the reservoir to be refilled while remaining in the scleral tunnel. Also, it may be noted that the reservoir could be inserted into a scleral tunnel without attachment to a scleral prosthesis, which would still allow for material delivery (possibly over an extended period of time).

In FIG. 3C, a scleral prosthesis (such as the prosthesis 100 or 150) can be inserted into a scleral tunnel 342 in a patients eye. A reservoir (such as any of the reservoirs 200-280) can be attached or secured to the scleral prosthesis by the connecting arms, although the reservoir itself has not been placed into a scleral tunnel. In this example, the reservoir sits above the sclera of the patient's eye and is not actually inserted into the scleral tissue. The reservoir could, for example, be placed below the conjunctiva of the patient's eye while remaining above the sclera or within a groove or incision created in the sclera (such as by using a laser or scalpel). In this example, the scleral prosthesis and/or the reservoir could be used to release medication or other material(s) into the patient's eye.

In FIG. 3D, a scleral prosthesis (such as the prosthesis 100 or 150) can be inserted into a scleral tunnel 362 in a patients eye. A reservoir (such as any of the reservoirs 200-280) can be inserted into a separate scleral tunnel 364 in the patient's eye and is attached or secured to the scleral prosthesis by the connecting arms. The scleral prosthesis and the reservoir in FIG. 3D are placed end to end, unlike the example shown in FIG. 3B where they are placed side by side. This illustrates that the scleral prosthesis and the reservoir could be used in a wide variety of configurations. Among other things, this may allow the scleral prosthesis and the reservoir to each be located in the most appropriate location in a patient's eye.

In FIG. 3E, a hypodermic needle 382 is used to refill a reservoir, which has been attached or secured to a scleral prosthesis. In some medical treatments (such as glaucoma treatments), the hypodermic needle 382 might ordinarily be used to inject medication or other material(s) directly into a patient's eye, and the injections could be repeated multiple times per year. This is clearly undesirable since it can cause much discomfort and worry to the patient. However, the implantation of the scleral prosthesis (with or without the reservoir) into the patient's eye could represent a one-time surgical procedure, which may be more acceptable to the patient. Moreover, once implanted, the scleral prosthesis or the reservoir could be repeatedly refilled with medication or other material(s), without requiring direct injection of the medication or other material(s) into the patient's eye. This may therefore cause little or no pain to the patient, increasing the likelihood of acceptance by the patient.

While various ones of these figures have illustrated the use of a scleral prosthesis and a reservoir, it may be noted that two reservoirs could be put into one or separate scleral tunnels (either end-to-end, side-by-side, or in another configuration) and connected by arms or other attachments. The arms or attachments and/or the opposing reservoirs could provide sufficient fixation to secure both reservoirs in place. Similarly, one of the reservoirs could be placed in a scleral tunnel, the other reservoir could be placed on the surface of the sclera, and arms or other attachments could provide sufficient fixation to secure both reservoirs in place. One or both of the reservoirs could be used to release medication or other material(s) into the patient's eye in either of these configurations.

Although FIGS. 3A through 3E illustrate examples of the uses of a scleral prosthesis (with and without a reservoir), various changes may be made to FIGS. 3A through 3E. For example, the scleral prosthesis and the reservoir could be used in any other suitable configuration. Also, any other suitable device or technique could be used to refill the scleral prosthesis or the reservoir.

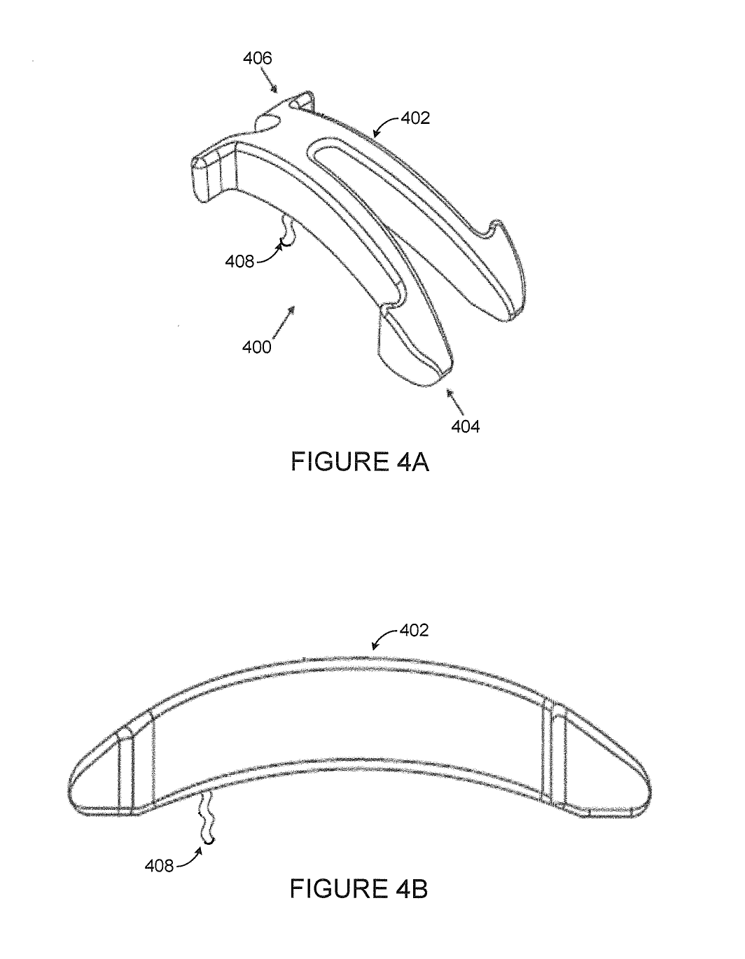

FIGS. 4A and 4B illustrate another example scleral prosthesis 400 in accordance with this disclosure. As shown in FIG. 4A, the scleral prosthesis 400 has a main body 402 with two opposing ends 404-406. Both ends 404-406 of the body 402 are split or divided into multiple legs or portions. The body 402 could include an internal cavity for storing medication or other material(s), and/or the prosthesis 400 could include an insert that is placed between the legs of the end 404 and that has an internal cavity for storing medication or other material(s). The prosthesis 400 could also be coupled to a reservoir that stores (and optionally releases) medication or other material(s).

The scleral prosthesis 400 in this example also includes a protrusion 408 that extends below the scleral prosthesis 400. The protrusion 408 represents a passageway through which medication or other material(s) could be delivered to an area below the scleral prosthesis 400. The protrusion 408 could have any suitable form and extend to any suitable distance below the scleral prosthesis 400. As particular examples, the protrusion 408 could represent a tube, prong, port, filament, or other suitable flexible or non-flexible passageway. While shown as being positioned on the body 402 of the scleral prosthesis, the protrusion 408 could be located on an insert placed between the portions forming the end 404 of the prosthesis 400.

Although FIGS. 4A and 4B illustrate another example of a scleral prosthesis 400, various changes may be made to FIGS. 4A and 4B. For example, any other suitable prosthesis could include a protrusion for delivering medication or other material(s) under the prosthesis. Also, multiple protrusions could be used with a single scleral prosthesis, or one or more protrusions could be used with a reservoir.

Note that while the scleral prostheses and some of the reservoirs described above have been described as being placed into the scleral tissue of an eye, the scleral prostheses and reservoirs described above could be used in any suitable manner. For example, a scleral prosthesis or reservoir can be placed entirely in scleral tissue of an eye or be placed under the sclera. If placed under the sclera, the prosthesis or reservoir could be placed on top of or ported to the ciliary body of the eye. The use of a protrusion to deliver material under the sclera could be useful since scleral elution often requires lipophilicity (since the sclera is a lipid barrier). A passageway to the ciliary body or other inner location of an eye allows almost any drug or other material to be delivered to the ciliary body or other location inside the eye. Other example locations for material delivery within the eye could include the suprachoroidal space (between the ciliary body and the sclera) and Schlemn's canal and the associated venous structures.

Any suitable medication(s) or other material(s) could be distributed using the prostheses 100 and 150. For example, medications could include drugs used to treat presbyopia, glaucoma, ocular hypertension, elevated intraocular pressure, macular degeneration, diabetic retinopathy, inflammatory and non-inflammatory ischemic optic neuropathy, central retinal vein occlusion, vasculitis, uveitis, inflammatory disorders affecting the posterior segment of the eye, or other eye disorders or diseases.

The medications could also include drugs used to treat eye-related cancers and inflammation of the eye (such as uveitis). As particular examples, the prostheses 100 and 150 could be used to distribute glaucoma drugs such as ACECORTAVE ACETATE, wet macular degeneration drugs such as ANECORTAVE ACETATE, peptide molecules such as TGF beta antagonists or other peptide molecules that are otherwise difficult to deliver and that could antagonize wound healing with other types of surgery, and long-term steroid therapy drugs. One possible advantage of these types of devices is that they may be used to deliver large molecules or peptides to the eye that would not enter the eye with topical administration. In fact, a large number of desirable drugs cannot enter the eye due to the size of the molecules, and these devices help to overcome that obstruction. As a particular example, anti-Vascular Endothelial Growth Factors (VEGF) drugs, such as AVASTIN, LUCENTIS, EYELEA, and other drugs being developed, could be eluted.

One or more medications could also be delivered using these devices and then activated in any suitable manner. For example, multiple medications could be released and activate each other. As another example, one or more medications can be released and then activated using a laser.

These devices may also be useful in the delivery of viral vectors for effecting drug therapy and gene therapy in the eye. Some viral vectors may need to be present for a long time, and these devices can result in sustained exposure. A particular example of this is adenoviral gene therapy since it requires multiple exposures.

In addition, these devices provide a mechanism for the sustained delivery of neuro-protective agents to the optic nerve. Some neuro-protective agents are lipophilic and cannot be administered either orally or as drops.

Various non-drug materials could also be administered using a scleral prosthesis or reservoir. For example, common therapies in molecular biology include silencing RNA (siRNA) therapies, which often involve repeated intravitreal injections.

Materials used for siRNA therapies often cannot be used intravenously or topically. As a particular example, one promising optic nerve protection drug is a silencing RNA therapy for CASPASE 2. A prosthesis or reservoir in or under the sclera could eliminate the need for repeated injections.

Another candidate for elution is cytokines or other derived peptides, which have an enormous number of biological effects. While only very small peptides typically work when applied topically, very large peptides or other materials can be delivered with an implant or reservoir in or under the sclera.

One particular example use of a scleral prosthesis or reservoir is for delivery of the medications needed after a cataract surgery. These medications can include a steroid, an antibiotic, and a nonsteroidal medication. Scleral delivery of these medications can be beneficial in various ways.

Another particular example use of a scleral prosthesis or reservoir is for delivery of chronic anti-metabolic and targeted anti-fibroblast medications, such as low-dose five floruracil or mitomycin C, for glaucoma surgery. These agents are typically injected or applied into a surgical site to limit the growth of fibroblasts. Scleral delivery of these medications can again be beneficial in various ways.

As noted above, a scleral prosthesis or reservoir could be used to deliver material into the sclera or other part of the eye. When placed in the sclera, the scleral prosthesis or reservoir could elute material into the sclera above, below, or laterally with respect to the scleral prosthesis or reservoir. If placed low enough within the sclera or under the sclera, the scleral prosthesis or reservoir could elute material into the ciliary body or other portion of the eye. The ciliary body can be a spongy structure in this vicinity and easily take up lipids, peptides, and hydrophilic materials.

A scleral prosthesis or reservoir could include any number of additional features depending on the implementation. The following represents several examples of additional features that could be used. A prosthesis or reservoir could contain a weakened center to facilitate its eventual removal. A prosthesis or reservoir could be lightly-colored so that its position can be easily determined for removal. A prosthesis or reservoir could have colored rings in order to code for the contents (medication or other material) contained within the prosthesis or reservoir. A prosthesis or reservoir could be recharged by a cannula inserted into the prosthesis or reservoir through a self-healing membrane, or the conjunctiva of the eye over the prosthesis or reservoir could be relied upon to heal and reseal the prosthesis or reservoir (such as if a 30-gauge cannula is used). A biological membrane such as an amniotic membrane or a reinforced amniotic membrane could also be used with a prosthesis or reservoir.

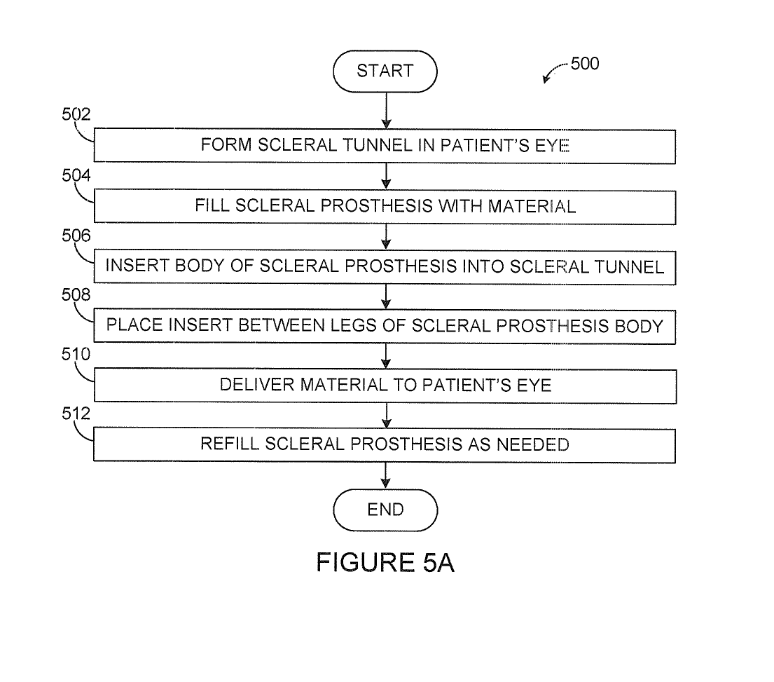

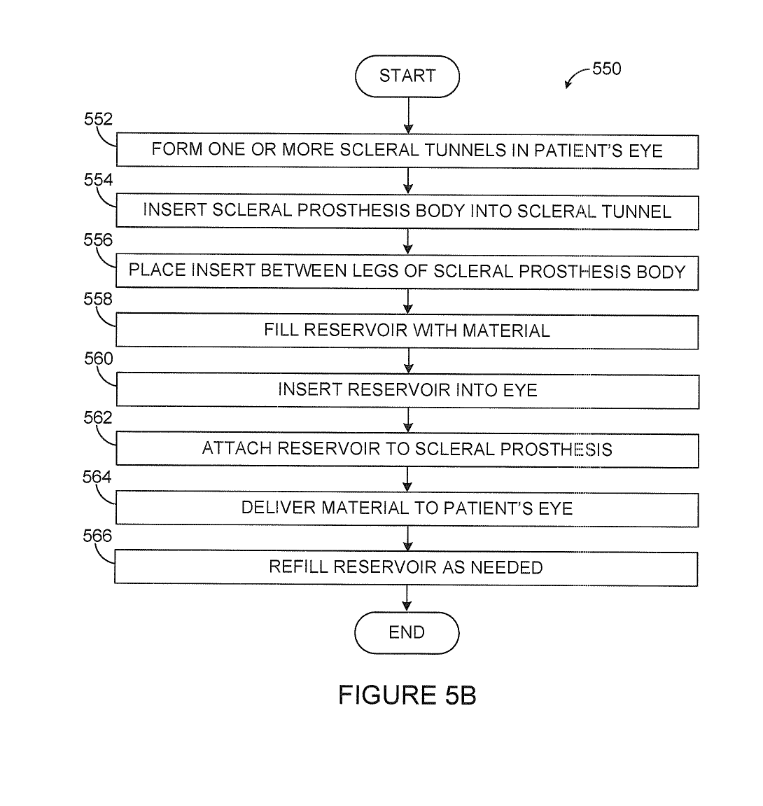

FIGS. 5A and 5B illustrate example methods for inserting and using a scleral prosthesis in a patient's eye in accordance with this disclosure. The embodiments of the methods shown in FIGS. 5A and 5B are for illustration only. Other embodiments of the methods could be used without departing from the scope of this disclosure.

FIG. 5A illustrates an example method 500 for placing a scleral prosthesis (without a reservoir) into a patient's eye. In FIG. 5A, a scleral tunnel is formed in the patient's eye at step 502. This could include, for example, using a tool with a straight or curved cutting blade or a specialized laser to form the scleral tunnel.

A scleral prosthesis is filled with one or more materials at step 504. This may include, for example, using a hypodermic needle to place medication or other material(s) into the insert 112 of the scleral prosthesis 100 or into the body 152 of the scleral prosthesis 150. Of course, medication or other material(s) could be placed in both the body and the insert of a scleral prosthesis, or no medication or other material(s) could be placed in the body or the insert until the prosthesis has been implanted in the patient's eye.

The body of the scleral prosthesis is inserted into the scleral tunnel at step 506. This could include, for example, pushing or pulling the scleral prosthesis body into the scleral tunnel. As a particular example, this could include pushing the legs of the prosthesis body together and pulling the scleral prosthesis body into the scleral tunnel. Once in the proper position, the legs of the scleral prosthesis body can be released, allowing the legs to return to a wider separation.

An insert is placed between the legs of the scleral prosthesis body at step 508. This could include, for example, locking the insert between the portions 108a-108b of the scleral prosthesis body 102 or between the portions of the scleral prosthesis body 152. The insert helps to maintain separation of the legs of the scleral prosthesis body. Among other things, this may help to provide a greater degree of stabilization for the scleral prosthesis.

At this point, the scleral prosthesis may remain in the patient's eye and deliver material to the patient's eye at step 510. This may include, for example, one or more membranes or other eluting surfaces of the scleral prosthesis providing one or more medications or other material(s) to the patient's eye. The medication(s) or other material(s) can be delivered slowly over time, which may be useful or necessary for treatments such as for glaucoma or macular degeneration.

The scleral prosthesis can be refilled with material as needed at step 512. This could include, for example, using a hypodermic needle to refill the scleral prosthesis body or insert with medication or other material(s). The medication or other material(s) could be constant or vary over time, such as when different drugs are used to treat the same eye disorder or disease or different eye disorders or diseases. It may be noted that refilling the scleral prosthesis may not be required, such as when the scleral prosthesis or the prior medication or other material(s) delivered to the patient's eye resolves the eye disorder or disease being treated.

FIG. 5B illustrates an example method 550 for placing a scleral prosthesis (with a reservoir) into a patient's eye. In FIG. 5B, one or more scleral tunnels are formed in the patient's eye at step 552. This could include, for example, using a tool with a straight or curved cutting blade or a specialized laser to form each scleral tunnel. If multiple scleral tunnels are formed, the tunnels could be formed side by side, end to end, or in any other suitable configuration.

A body of a scleral prosthesis is inserted into one of the scleral tunnels at step 554. This could include, for example, pushing or pulling the scleral prosthesis body into the scleral tunnel. An insert is placed between the legs of the scleral prosthesis body at step 556. This could include, for example, locking the insert between the portions 108a-108b of the scleral prosthesis body 102 or between the portions of the scleral prosthesis body 152.

A reservoir is filled with one or more medications or other material(s) at step 558. This may include, for example, using a hypodermic needle to place medication or other material(s) into the reservoir. Of course, no medication or other material(s) may be placed into the reservoir until after the reservoir is inserted into a scleral tunnel or otherwise secured in or on the patient's eye.

The reservoir is inserted into the patient's eye at step 560. This could include, for example, pushing or pulling the reservoir into a scleral tunnel, which could be the same scleral tunnel in which the prosthesis is placed or a different scleral tunnel. The reservoir is attached or secured to the scleral prosthesis at step 562. This could include, for example, using connecting arms to attach or secure the reservoir to the scleral prosthesis 100 or 150. The connecting arms could form part of the reservoir or part of the scleral prosthesis, or the connecting arms could represent separate components that are coupled to both the reservoir and the scleral prosthesis. The connecting arms could be coupled to the reservoir and/or the scleral prosthesis in any suitable manner, such as by using an adhesive, welding, fusing, or any other suitable technique.

At this point, the scleral prosthesis and the reservoir may remain in the patient's eye and deliver medication or other material(s) to the patient's eye at step 564. This may include, for example, one or more membranes or other eluting surfaces of the scleral prosthesis and/or the reservoir providing one or more medications or other material(s) to the patient's eye.

The reservoir can be refilled with medication or other material(s) as needed at step 566. This could include, for example, using a hypodermic needle to refill the reservoir with medication or other material(s). The medication or other material(s) could be constant or vary over time. Also, it may be noted that refilling the reservoir may not be required.

During either of these methods, the patient's eye could be secured in place in any suitable manner. For example, the patient's eye could be secured using one of the devices disclosed in U.S. Patent Publication No. 2008/0091224 or U.S. Patent Publication No. 2012/0226107 (both of which are hereby incorporated by reference). Moreover, the scleral prosthesis and/or the reservoir could be inserted into a patient's eye in any suitable location(s). As particular examples, the scleral prosthesis and/or the reservoir could be placed in a location proximate to the trabecular meshwork of the eye for the treatment of glaucoma through treatment to the trabecular meshwork (where aqueous achieves egress) or the ciliary body (where aqueous is made).

Although FIGS. 5A and 5B illustrate examples of methods for inserting and using a scleral prosthesis in a patient's eye, various changes may be made to FIGS. 5A and 5B. For example, while shown as a series of steps, various steps in FIGS. 5A and 5B could overlap, occur in parallel, occur in a different order, or occur multiple times. Also, various steps in FIGS. 5A and 5B could be omitted depending on the implementation. As a particular example, in FIGS. 5A and 5B, an insert may not be used with the scleral prosthesis, and steps 508 and 556 could be omitted. Further, any suitable number of scleral prostheses (and optionally any suitable number of reservoirs) could be implanted into a patient's eye. As an example, four scleral prostheses could be implanted into a patient's eye, and one, some, or all of the scleral prostheses could be used for material delivery. As another example, four scleral prostheses could be implanted into a patient's eye, and a reservoir could be attached to one, some, or all of the scleral prostheses.

In addition, FIG. 5A has illustrated the implantation of a scleral prosthesis, and FIG. 5B has illustrated the implantation of a scleral prosthesis and a reservoir. However, it may be noted that the method 500 could be modified to involve the implantation of only a reservoir, which is used in a stand-alone manner and not in conjunction with a scleral prosthesis. Similarly, the method 550 could be modified to involve the implantation of two reservoirs that are used together, without being used in conjunction with a scleral prosthesis. In fact, any number of scleral prostheses and/or reservoirs, as well as any combination of scleral prostheses and/or reservoirs, could be used in a patient's eye to provide any desired treatment to the patient's eye.

It may be advantageous to set forth definitions of certain words and phrases used throughout this patent document. The terms “include” and “comprise,” as well as derivatives thereof, mean inclusion without limitation. The term “or” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. A scleral prosthesis comprising:
an elongated body that is narrower in a middle and wider at opposing first and second ends, the body comprising multiple first portions that form the first end of the body, the first portions separated lengthwise along the body;
wherein the first portions are biased to maintain separation without external interference but are configured to be pushed towards each other in order to narrow a width of the first end.

2. The scleral prosthesis of claim 1, wherein the body further comprises:
a cavity configured to receive a material; and
an eluting surface configured to release the material from the cavity.

3. The scleral prosthesis of claim 2, wherein the body further comprises a refill hole configured to receive the material for storage in the cavity.

4. The scleral prosthesis of claim 3, wherein the refill hole is located at one of the first end and the second end of the scleral prosthesis and remains accessible after the scleral prosthesis is implanted into a patient's eye.

5. The scleral prosthesis of claim 1, wherein the body further comprises:
a cavity configured to receive a material; and
a protrusion that extends away from the scleral prosthesis, the protrusion configured to release the material from the cavity.

6. The scleral prosthesis of claim 1, wherein the body further comprises second portions that form the second end of the body, the second portions separated lengthwise along the body.

7. The scleral prosthesis of claim 6, wherein the second portions are separated along less than a quarter of a total length of the body.

8. The scleral prosthesis of claim 1, wherein the first portions are separated along at least about 50% of a total length of the body.

9. A scleral prosthesis comprising:
an elongated body that is narrower in a middle and wider at opposing first and second ends, wherein the body comprises:
multiple first portions that form the first end of the body, the first portions separated lengthwise along the body, wherein the first portions are biased to maintain separation without external interference but are configured to be pushed towards each other in order to narrow a width of the first end; and
an insert configured to be placed between the first portions to maintain the separation of the first portions.

10. The scleral prosthesis of claim 9, wherein at least one of the body and the insert comprises:
a cavity configured to receive a material; and
an eluting surface configured to release the material from the cavity.

11. The scleral prosthesis of claim 10, wherein at least one of the body and the insert further comprises a refill hole configured to receive the material for storage in the cavity.

12. The scleral prosthesis of claim 11, wherein the refill hole is located at one of the first end and the second end of the scleral prosthesis and remains accessible after the scleral prosthesis is implanted into a patient's eye.

13. The scleral prosthesis of claim 9, wherein at least one of the body and the insert comprises:
a cavity configured to receive a material; and
a protrusion that extends away from the scleral prosthesis, the protrusion configured to release the material from the cavity.

14. The scleral prosthesis of claim 13, wherein the scleral prosthesis is configured so that, when implanted in scleral tissue of a patient's eye, the protrusion releases the material from the cavity into a ciliary body of the patient's eye.

15. The scleral prosthesis of claim 9, wherein the body further comprises second portions that form the second end of the body, the second portions separated lengthwise along the body.

16. The scleral prosthesis of claim 15, wherein the second portions are separated along less than a quarter of a total length of the body.

17. The scleral prosthesis of claim 9, wherein the first portions are separated along at least about 50% of a total length of the body.

18. A system comprising:
a scleral prosthesis configured to be implanted into scleral tissue of a patient's eye; and
a reservoir configured to be coupled to the scleral prosthesis, the reservoir comprising a cavity configured to receive a material;
wherein at least one of the scleral prosthesis and the reservoir is configured to release the material into the patient's eye.

19. The system of claim 18, wherein at least one of the scleral prosthesis and the reservoir comprises a protrusion that extends away from the scleral prosthesis or reservoir, the protrusion configured to release the material from the cavity.

20. The system of claim 18, wherein:
the scleral prosthesis comprises an elongated body that is narrower in a middle and wider at opposing first and second ends, the body comprising multiple first portions that form the first end of the body, the first portions separated lengthwise along the body; and
the first portions are biased to maintain separation without external interference but are configured to be pushed towards each other in order to narrow a width of the first end.

21. The system of claim 20, wherein the scleral prosthesis further comprises an insert configured to be placed between the first portions to maintain the separation of the first portions.

22. The system of claim 18, wherein the reservoir comprises a refill hole configured to receive the material for storage in the cavity.
